(19) United States
(12) Patent Application Publication
(10) Pub. No.: US 2014/0288073 A1
(43) Pub. Date: Sep. 25, 2014

(54) Method of Treating Gastrointestinal Stromal Tumors

(71) Applicant: NOVARTIS AG, Basel (CH)

(72) Inventors: John E. Monahan, Walpole, MA (US); Fang Li, Arlington, MA (US)

(73) Assignee: Novartis AG, Basel (CH)

(21) Appl. No.: 14/353,186

(22) PCT Filed: Oct. 24, 2012
(86) PCT No.: PCT/US2012/061532

§ 371 Date: Apr. 21, 2014

Related U.S. Application Data
(60) Provisional application No. 61/552,633, filed on Oct. 28, 2011.

(51) Int. Cl. A61K 31/506; A61K 31/4439; A61K 31/5377; A61K 31/4745; A61K 31/496

(52) U.S. Cl. CPC A61K 31/506; A61K 31/4745; A61K 31/496; A61K 31/5377; A61K 31/4439
USPC 514/235.8; 514/252.18

(57) ABSTRACT

The present invention relates to a method of treating gastrointestinal stromal tumors (GIST), especially GIST, which is progressing after imatinib therapy or after imatinib and sunitinib therapy, using a combination comprising (a) a c-kit inhibitor and (b) a PI3K inhibitor or FGFR inhibitor.

The present invention relates to a method of treating gastrointestinal stromal tumors (GIST) in a human patient population using a combination comprising (a) a c-kit inhibitor and (b) a PI3K inhibitor or FGFR inhibitor.

GIST are the most frequent mesenchymal tumors of the gastrointestinal tract. These tumors are thought to arise from the interstitial cells of Cajal, which compose the myenteric plexus found in the stomach and bowel. Primary GIST most frequently occur in the stomach (50-60%), small bowel (20-30%), and large bowel (10%), with the esophagus, mesentery, omentum, and retroperitoneum accounting for the remaining cases. On the basis of population-based incidence rates in Sweden, it has been estimated that approximately 5000 new cases of GIST are diagnosed each year in the US. GIST predominantly occur in middle-aged and older people, with a median onset age of approximately 60 years and no apparent gender preference.

GIST may display a variety of phenotypic features, many of which correlate with patient prognosis. Thus, a consensus meeting emphasized tumor size and mitotic index for risk stratification of primary GIST, with such risk being correlated with tumor recurrence. At the present time, risk stratification based on pathologic criteria is preferable to the use of such terms as benign or malignant GIST. Patients with primary gastric GIST seem to fare slightly better than those with intestinal tumors. GIST have a tendency to recur both locally and in the form of peritoneal and liver metastases, with lymph-node metastases being infrequent. Surgical resection is the mainstay of therapy for primary GIST, and the disease is typically refractory to cytotoxic chemotherapy. The diagnosis of GIST has been facilitated by the discovery that these tumors stain positively with an immunohistochemical marker (CD117) previously used to stain the interstitial cells of Cajal. The antibody used in the immunohistochemical reaction recognizes the extracellular domain of the stem-cell factor receptor, KIT. Currently, KIT expression is a major diagnostic criterion for GIST, and few other KIT-positive mesenchymal tumors of the gastrointestinal tract are likely to be confused with GIST; notable exceptions include metastatic melanoma and malignant vascular tumors. Approximately 95% of GIST stain positively for CD117. In most of these cases, somatic mutations can be found in the gene encoding the KIT protein, typically in exons 11, 9 and 13. These mutations confer a gain of function to the receptor, which becomes constitutively activated regardless of the presence of ligand.

The mainstay of therapy for patients with primary GIST is surgical resection. However, surgery alone is generally not curative; the 5-year disease specific survival is reported to be 54%. Recurrence rates exceeding 50% within 2 years of resection of primary GIST and approximating 90% after re-excision, underscored the need for effective postoperative treatment.

Imatinib received worldwide approval for the treatment of adult patients with KIT-positive (CD117) and unresectable and/or metastatic GIST and dramatically changed the prognosis for such patients by prolonging the overall and the progression-free-survival (PFS) and increasing the 5-year survival rate. Imatinib at doses ranging from 400 mg/day to 800 mg/day is used worldwide for the treatment of patients with unresectable and/or metastatic KIT-positive GIST. In addition, imatinib 800 mg/day significantly improves progression-free survival (PFS) in patients with advanced GIST harboring KIT exon 9 mutations compared to 400 mg/day.

As a result of the efficacy of imatinib for the treatment of patients with unresectable and/or metatastatic GIST, a double-blind, randomized phase III study (ACOSOGZ9001) was conducted to determine whether adjuvant treatment of adult patients with GIST following complete resection with 400 mg/day of imatinib for 12 months improved recurrence-free survival (RFS) compared with placebo. The results of the study indicated that treatment with imatinib significantly prolonged RFS. Based upon these data, imatinib at a dose of 400 mg/day was approved worldwide for adjuvant treatment of adult patients following resection of GIST. Results from SSGXVIII/AIO, a Phase III multicenter, open-label, randomized study to assess the efficacy and safety of 400 mg imatinib once daily over 12 months or 36 months in GIST patients following surgery, and who were estimated to be at high risk of disease recurrence are now available. The study data confirm that 36 months of adjuvant therapy with imatinib is well tolerated and superior to 12 months in prolonging RFS and overall survival in patients with GIST following surgical resection.

Despite the efficacy of imatinib, the treatment of metastatic GIST remains an area of unmet medical need with more than 50% of patients with advanced GIST progressing after 2 years of imatinib first line therapy.

Sunitinib (Sutent®; Pfizer), approved for use following progression on imatinib, is the only agent other than Glivec to be approved for the treatment of advanced unresectable GIST. The agent has demonstrated efficacy in patients who have progressed on imatinib therapy. However, Sutent's tolerability profile is a limiting factor for long-term use in GIST.

It was now found that combining a KIT inhibitor and inhibitors that target the survival pathways in GIST can produce a greater therapeutic effect than that obtained by administration of a KIT inhibitor alone.

As shown herein, the FGF2 growth factor and its receptor FGFR1 are over-expressed in primary GIST tissue, suggesting that FGFR pathway could be a survival pathway activated in GIST. FGFR1, but not FGF2, is over-expressed in GIST cell lines. However, the FGFR signaling pathway is activated in GIST cell lines in the presence of exogenous FGF2. In addition, GIST cell lines are less sensitive to the treatment of KIT inhibitors in the presence of added FGF2. Combination of FGFR inhibitors with KIT inhibitors produces strong synergistic activity and significantly improved efficacy in the presence of FGF2 in GIST cells, suggesting that a combination comprising an FGFR inhibitor and a KIT inhibitor can improve the efficacy of the current treatment strategies in GIST.

In a broader sense, the present invention provides a method of treating GIST, preferably GIST not harboring any KIT mutations, by administering to a patient in need thereof a therapeutically effective amount of a FGFR inhibitor.

Furthermore, based on observations in GIST cell lines it was now surprisingly found that patients with GIST progressing after imatinib first line therapy, might be treated successfully with a combination comprising (a) a c-kit inhibitor and (b) a PI3K inhibitor.

Furthermore, it is concluded that patients with GIST progressing after consecutive therapy with imatinib and sunitinib can be treated successfully with a combination comprising (a) a c-kit inhibitor and (b) a PI3K inhibitor.

Hence, the present invention provides a method for treating GIST in a human patient progressing after imatinib therapy or consecutive imatinib and sunitinib therapy, comprising co-administration to said patient, e.g., concomitantly or in sequence, of a therapeutically effective amount of (a) a c-kit inhibitor and (b) a PI3K inhibitor or FGFR inhibitor. More broadly, the present invention provides a method for treating GIST in a human patient in need thereof, comprising co-administration to said patient, e.g., concomitantly or in sequence, of a therapeutically effective amount of (a) a c-kit inhibitor and (b) a PI3K inhibitor or FGFR inhibitor.

In a further aspect, the present invention relates to the use of a combination comprising (a) a c-kit inhibitor and (b) a PI3K inhibitor or FGFR inhibitor for the manufacture of a medicament for the treatment of GIST, especially GIST progressing after imatinib first line therapy.

A further aspect of the invention relates to a combination comprising (a) a c-kit inhibitor and (b) a PI3K inhibitor or FGFR inhibitor for the treatment of GIST, especially GIST progressing after imatinib therapy or GIST progressing after imatinib and sunitinib therapy.

### SHORT DESCRIPTION OF THE FIGURES

FIG. 1: FGF2 and FGFR1 are highly expressed in primary GISTs. Raw data (CEL files) of the expression profiles for 30,094 primary tumors were normalized by MAS5 algorithm using 150 as the target value.

FIG. 2: FGF2 expression is substantially higher in KIT-positive primary gastrointestinal stromal tumors (GISTs) than in other human primary tumor tissues. GAPDH Western blot is shown as a loading control.

FIG. 3: FGFR pathway is activated in GIST cell lines in the presence of various concentrations of added FGF2. FRS2 Tyr-Phosphorylation was used as the readout of FGFR signaling activation and measured by Western blot in the GIST cell lines. Total FRS2 level is shown as the loading control.

FIG. 4: GIST cell lines are less sensitive to the treatment of a KIT inhibitor AMN107 (nilotinib) in the presence of added FGF2. GIST-T1 and GIST882 cell lines were treated with AMN107 for 3 days with serial dilutions of the KIT inhibitor AMN107 in the absence or presence of 50 ng/ml, 25 ng/ml, 12 ng/ml FGF2. Relative cell growth was measured by Cell Titer Glo assay and expressed as a percentage of DMSO-treated cells.

FIG. 5: Combination effect of imatinib plus BGJ398 in GIST-T1 and GIST882 in the absence and of presence of 20 ng/ml FGF2. The left panels show the percent inhibition relative to DMSO-treated cells for each single agent and combination treatments. Increasing concentrations of imatinib (CGP057148B) are shown along the left column from bottom to top and increasing concentrations of BGJ398 along the bottom row from left to right. The middle panels show the excess inhibition for each point in the left panels. Excess inhibition was determined based on the Loewe synergy model that measures the effect on growth relative to what should be expected if the two drugs only function additively. Positive numbers indicate synergy, and negative numbers antagonism. The right panels are the isobolograms that display the interactions between the two compounds. The red straight lines connecting the doses of imatinib and BGJ398 represent the additive effect. Blue curves that lie below and to the left of the straight lines represent synergism.

FIG. 6: Combination effect of nilotinib plus BGJ398 in GIST cell lines in the presence of 20 ng/ml FGF2.

The expression “c-kit inhibitor” as used herein includes, but is not limited to, 4-(4-methylpiperazin-1-ylmethyl)-N-[4-methyl-3-(4-(pyridin-3-yl)pyrimidin-2-ylamino)phenyl]-benzamide (Imatinib), 4-methyl-3-[[4-(3-pyridinyl)-2-pyrimidinyl]amino]-N-[5-(4-methyl-1H-imidazol-1-yl)-3-(trifluoromethyl)phenyl]benzamide (Nilotinib), masitinib, sunitinib, sorafenib, regorafeinib, motesanib, and, respectively, the pharmaceutically acceptable salts thereof.

In a preferred embodiment the c-kit inhibitor employed is Imatinib. Imatinib is specifically disclosed in the U.S. Pat. No. 5,521,184, the subject-matter of which is hereby incorporated into the present application by reference. Imatinib can also be prepared in accordance with the processes disclosed in WO03/066613. For the purpose of the present invention, Imatinib is preferably applied in the form of its mono-mesylate salt. Imatinib mono-mesylate can be prepared in accordance with the processes disclosed in U.S. Pat. No. 6,894,051. Comprised by the present invention are likewise the corresponding polymorphs, e.g. crystal modifications, which are disclosed in U.S. Pat. No. 6,894,051.

In a further preferred embodiment of the method described herein the mono-mesylate salt of Imatinib is administered orally in dosage forms as described in U.S. Pat. No. 5,521,184, U.S. Pat. No. 6,894,051 or US 2005-0267125. The mesylate salt of Imatinib is marketed under the brand Glivec® (Gleevec®). A preferred oral daily dosage of Imatinib is 200-600 mg, in particular 400 mg/day, administered as a single dose or divided into multiple doses, such as twice daily dosing.

In a further preferred embodiment of the present invention, the c-kit inhibitor employed is Nilotinib. Nilotinib and the process for its manufacture are disclosed in WO 04/005281, which is hereby incorporated into the present application by reference. Pharmaceutically acceptable salts of Nilotinib are especially those disclosed in WO2007/015871. For the purpose of the present invention, Nilotinib is preferably applied in the form of its mono-hydrochloride monohydrate salt. WO2007/015870 discloses certain polymorphs of nilotinib and its pharmaceutically acceptable salts useful for the present invention.

In a further preferred embodiment of the method described herein the mono-hydrochloride salt of Nilotinib is administered orally in dosage forms as described in WO2008/037716. The mono-hydrochloride salt of Nilotinib is marketed under the brand Tasigna®. A preferred oral daily dosage of Nilotinib is 200-1200 mg, e.g. 800 mg, administered as a single dose or divided into multiple doses, such as twice daily dosing.

The phosphatidylinositol 3-kinases (PI3Ks) are a family of lipid kinases which phosphorylate the 3′-OH group of phosphatidylinositols in the lumen side of the cell membrane, and are involved in the regulation of a wide range of cellular processes. In response to lipid phosphorylation (PIP₂ to PIP₃) various signaling proteins, including the protein serine-threonine kinase AKT, are recruited to the plasma membrane, where they become activated and initiate a signal transduction cascade.

There are three classes of PI3Ks (I-III), and currently 8 members of the family are known. The class I enzymes consist of heterodimers having a regulatory (p85) domain and a catalytic (p110) subunit, of which there are four isoforms: p110α, p110β, p110δ and p110γ. The α and β isoforms are ubiquitously expressed; α is linked upstream mainly to receptor tyrosine kinases, whereas β can mediate signals from both G-protein-coupled receptors and from receptor tyrosine kinases. The δ and γ isoforms are expressed primarily in lymphocytes and play important roles in the regulation of immune responses. The γ isoform is also highly expressed in GIST. However, the function of γ isoform in GIST is still unknown.

A gain of function in PI3K signaling is common in many types of human cancer and include inactivation of the PTEN tumor suppressor gene, amplification/overexpression or activating mutations of some receptor tyrosine kinases (e.g. erbB3, erbB2, EGFR), amplification of genomic regions containing AKT, amplification of PIK3CA (the gene encoding p110α) and mutations in p110α. More than 30% of various solid tumor types were recently found to contain mutations of PIK3CA. From these mutation frequencies, PIK3CA is one of the most commonly mutated genes identified in human cancers.

The expression “PI3K inhibitor” as used herein includes, but is not limited to those specified below,

WO2006/122806 describes imidazoquinoline derivatives, which have been described to inhibit the activity of lipid kinases, such as PI3-kinases. Specific imidazoquinoline derivatives which are suitable for the present invention, their preparation and suitable pharmaceutical formulations containing the same are described in WO2006/122806 and include compounds of formula I

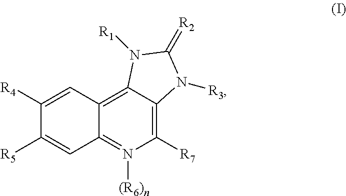

wherein
R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl; pyrazolyl; and triazolyl;

### R₂ is O or S;

R₃ is lower alkyl;
R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen; quinoxalinyl; or phenyl substituted with alkoxy R₅ is hydrogen or halogen;
n is 0 or 1;
R₆ is oxido; with the proviso that if n=1, the N-atom bearing the radical R₆ has a positive charge;
R₇ is hydrogen or amino;
or a tautomer thereof, or a pharmaceutically acceptable salt, or a hydrate or solvate thereof.

The radicals and symbols as used in the definition of a compound of formula I have the meanings as disclosed in WO2006/122806 which publication is hereby incorporated into the present application by reference.

A preferred compound of the present invention is a compound which is specifically described in WO2006/122806. A very preferred compound of the present invention is 2-methyl-2-[4-(3-methyl-2-oxo-8-quinolin-3-yl-2,3-dihydro-imidazo[4,5-c]quinolin-1-yl)-phenyl]-propionitrile and its monotosylate salt (COMPOUND A). The synthesis of 2-methyl-2-[4-(3-methyl-2-oxo-8-quinolin-3-yl-2,3-dihydro-imidazo[4,5-c]quinolin-1-yl)-phenyl]-propionitrile is for instance described in WO2006/122806 as Example 1. Another very preferred compound of the present invention is 8-(6-methoxy-pyridin-3-yl)-3-methyl-1-(4-piperazin-1-yl-3-trifluoromethyl-phenyl)-1,3-dihydro-imidazo[4,5-c]quinolin-2-one (COMPOUND B). The synthesis of 8-(6-methoxy-pyridin-3-yl)-3-methyl-1-(4-piperazin-1-yl-3-trifluoromethyl-phenyl)-1,3-dihydro-imidazo[4,5-c]quinolin-2-one is for instance described in WO2006/122806 as Example 86.

WO07/084,786 describes pyrimidine derivatives, which have been found the activity of lipid kinases, such as PI3-kinases. Specific pyrimidine derivatives which are suitable for the present invention, their preparation and suitable pharmaceutical formulations containing the same are described in WO07/084,786 and include compounds of formula I

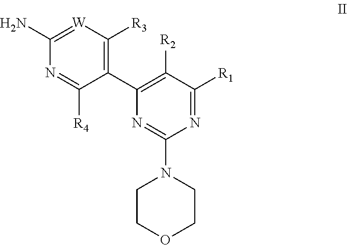

or a stereoisomer, tautomer, or pharmaceutically acceptable salt thereof, wherein,
W is CR_{w} or N, wherein R_{w} is selected from the group consisting of
(1) hydrogen,
(2) cyano,
(3) halogen,
(4) methyl,
(5) trifluoromethyl,
(6) sulfonamido;
R₁ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,

### (12) —COR₁ₐ,

### (13) —CO₂R₁ₐ,

### (14) —CONR₁ₐR_{1b},

### (15) —NR₁ₐR_{1b},

### (16) —NR₁ₐCOR_{1b},

### (17) —NR₁ₐSO₂R_{1b},

### (18) —OCOR₁ₐ,

### (19) —OR₁ₐ,

### (20) —SR₁ₐ,

### (21) —SOR₁ₐ,

### (22) —SO₂R₁ₐ, and

### (23) —SO₂NR₁ₐR_{1b},

wherein R₁ₐ, and R_{1b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;
R₂ is selected from the group consisting
(1) hydrogen,
(2) cyano,
(3) nitro,
(4) halogen,
(5) hydroxy,
(6) amino,
(7) substituted and unsubstituted alkyl,

### (8) —COR₂ₐ, and

(9) —NR₂ₐCOR_{2b}, wherein R₂ₐ, and R_{2b} are independently selected from the group consisting of
(a) hydrogen, and
(b) substituted or unsubstituted alkyl; R₃ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,

### (12) —COR₃ₐ,

### (13) —NR₃ₐR_{3b},

### (14) —NR₃ₐCOR_{3b},

### (15) —NR₃ₐSO₂R_{3b},

### (16) —OR₃ₐ,

### (17) —SR₃ₐ,

### (18) —SOR₃ₐ,

### (19) —SO₂R₃ₐ, and

(20) —SO₂NR₃ₐR_{3b}, wherein R₃ₐ, and R_{3b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;
and R₄ is selected from the group consisting of
(1) hydrogen, and
(2) halogen.

The radicals and symbols as used in the definition of a compound of formula I have the meanings as disclosed in WO07/084,786 which publication is hereby incorporated into the present application by reference.

A preferred compound of the present invention is a compound which is specifically described in WO07/084,786. A very preferred compound of the present invention is 5-(2,6-di-morpholin-4-yl-pyrimidin-4-yl)-4-trifluoromethyl-pyridin-2-ylamine (COMPOUND C). The synthesis of 5-(2,6-di-morpholin-4-yl-pyrimidin-4-yl)-4-trifluoromethyl-pyridin-2-ylamine is described in WO07/084,786 as Example 10.

A further preferred PI3K inhibitor of the present invention is (S)-pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (COMPOUND D) or a pharmaceutically acceptable salt thereof. The synthesis of (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) is for instance described in WO 2010/029082 as Example 15.

The expression “FGFR inhibitor” as used herein includes, but is not limited to

(a) brivanib, intedanib, E-7080, ponatinib, SU-6668 and AZD-4547.
(b) the compounds disclosed in WO2009/141386, and
(c) WO2006/000420 (including 3-(2,6-dichloro-3,5-dimethoxy-phenyl)-1-{6-[4-(4-ethyl-piperazin-1-yl)-phenylamino]-pyrimid-4-yl}-1-methyl-urea monophosphate, BGJ398). BGJ398 is a pan-FGFR kinase inhibitor inhibiting FGFR 1-3 (IC50 between 3 and 7 nM).

The following aspects of the invention are of particular importance:
(1.) A method of treating GIST in a human patient comprising administering to the human patient in need thereof a dose effective against GIST of a combination (a) a c-kit inhibitor and (b) a PI3K inhibitor or FGFR inhibitor, or a pharmaceutically acceptable salt thereof, respectively, especially wherein the c-kit inhibitor is selected from imatinib, nilotinib and masitinib, or, respectively, a pharmaceutically acceptable salt thereof.
(2.) A method of treating GIST in a human patient comprising administering to the human patient in need thereof a dose effective against GIST, wherein the GIST is progressing after imatinib therapy or after imatinib and sunitinib therapy.
(3.) A combination comprising (a) a c-kit inhibitor and (b) a PI3K inhibitor or FGFR inhibitor or a pharmaceutically acceptable salt thereof, respectively, for the treatment of GIST.

For the purposes of the present invention, a combination comprising (a) a c-kit inhibitor and (b) a PI3K inhibitor or FGFR inhibitor is preferably selected from

(1) imatinib or a pharmaceutically acceptable salt thereof and COMPOUND A or a pharmaceutically acceptable salt thereof,
(2) imatinib or a pharmaceutically acceptable salt thereof and COMPOUND C or a pharmaceutically acceptable salt thereof,
(3) imatinib or a pharmaceutically acceptable salt thereof and COMPOUND D or a pharmaceutically acceptable salt thereof,
(4) masitinib or a pharmaceutically acceptable salt thereof and COMPOUND A or a pharmaceutically acceptable salt thereof,
(5) masitinib or a pharmaceutically acceptable salt thereof and COMPOUND C or a pharmaceutically acceptable salt thereof, and
(6) masitinib or a pharmaceutically acceptable salt thereof and COMPOUND D or a pharmaceutically acceptable salt thereof,
(7) imatinib or a pharmaceutically acceptable salt thereof and BGJ398 or a pharmaceutically acceptable salt thereof,
(8) masitinib or a pharmaceutically acceptable salt thereof and BGJ398 or a pharmaceutically acceptable salt thereof,
(9) nilotinib or a pharmaceutically acceptable salt thereof and BGJ398 or a pharmaceutically acceptable salt thereof,
(10) imatinib or a pharmaceutically acceptable salt thereof and FGFR inhibitor selected from brivanib, intedanib, E-7080, ponatinib, SU-6668 and AZD-4547.
(11) a c-KIT inhibitor selected from sunitinib, sorafenib, regorafenib, motesanib or a pharmaceutically acceptable salt thereof, respectively, and COMPOUND A or a pharmaceutically acceptable salt thereof,
(12) a c-KIT inhibitor selected from sunitinib, sorafenib, regorafenib, motesanib or a pharmaceutically acceptable salt thereof, respectively, and COMPOUND C or a pharmaceutically acceptable salt thereof,
(13) a c-KIT inhibitor selected from sunitinib, sorafenib, regorafenib, motesanib or a pharmaceutically acceptable salt thereof, respectively, and COMPOUND D or a pharmaceutically acceptable salt thereof, and
(14) a c-KIT inhibitor selected from sunitinib, sorafenib, regorafenib, motesanib or a pharmaceutically acceptable salt thereof, respectively, and BGJ398 or a pharmaceutically acceptable salt thereof.

For the purposes of the present invention, the PI3K inhibitor is preferably selected from 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, and (S)-pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), or, respectively, a pharmaceutically acceptable salt thereof.

The structure of the active agents identified by generic or trade names may be taken from the actual edition of the standard compendium “The Merck Index” or from databases, e.g. Patents International (e.g. IMS World Publications). The corresponding content thereof is hereby incorporated by reference.

Unless mentioned otherwise, the PI3K inhibitors, c-KIT inhibitors and FGFR inhibitors are used in a dosage as either specified in the product information of a product comprising such inhibitor for the treatment of a proliferative disorder, or, especially if such product information is not available, in a dosage which is determined in dose finding studies.

Suitable clinical studies in human patients are, for example, open label non-randomized, studies in patients with GIST progressing after imatinib first line therapy. Such studies prove in particular superiority of the claimed method of treatment compared to treatments with one of the components of the treatment schedule alone. The beneficial effects on GIST can be determined directly through the results of these studies (e.g. RFS or progression free survival—PFS) or by changes in the study design which are known as such to a person skilled in the art.

### EXAMPLES

The following Example illustrates the invention described above, but is not, however, intended to limit the scope of the invention in any way. Other test models known as such to the person skilled in the pertinent art can also determine the beneficial effects of the claimed invention.

### Example 1

### FGF receptor 1 (FGFR1) and FGF2 expression in primary GISTs

### Cell Lines and Culture

GIST882, GIST48 and GIST430 cell lines were obtained from the Brigham and Women's Hospital, Boston, Mass. GIST882 was established from an untreated human GIST with a homozygous missense mutation in KIT exon 13, encoding a K642E mutant KIT protein (Tuveson D A, Willis N A, et al. Oncogene 2001; 20: 5054-5058). GIST48 and GIST430 were established from GISTs that has progressed after initial clinical response to imatinib treatment (Bauer S, Yu L K, Demetri G D, Fletcher J A. Cancer Res 2006; 66: 9153-9161). GIST48 has a primary homozygous exon 11 missense mutation (V560D) and a secondary heterozygous exon 17 missense mutation (D820A). GIST430 has a primary heterozygous exon 11 in-frame deletion and a secondary heterozygous exon 13 missense mutation (V654A). GIST-T1 was obtained from Kochi Medical School, Kochi, Japan. It was established from a metastatic human GIST with a heterozygous deletion of 57 bases in exon 11 of KIT (Taguchi T, Sonobe H, Toyonaga S, et al. Lab Invest 2002; 82: 663-665).

GIST882 cells were cultured in RPMI-1640 (ATCC Catalog #30-2001) supplemented with 15% FBS and 1% L-glutamine, GIST48 cells in F10 (Gibcol/Invitrogen Catalog #11550-043) supplemented with 15% FBS, 0.5% Mito+(BD Bioscience Catalog #355006), 1% BPE (BD Bioscience/Fisher Catalog#354123) and 1% L-glutamine, GIST430 cells in IMEM (Gibco/Invitrogen Catalog #12440-053) supplemented with 15% FBS and 1% L-glutamine, and GIST-T1 cells in DMEM (Gibcol/Invitrogen Catalog #11965) supplemented with 10% FBS.

### Cell Viability Assay

Imatinib and BGJ398 were dissolved in DMSO as a 10 mM stock, and subsequently diluted with media to make a series of working solutions at concentrations (μM) of 0, 0.02, 0.05, 0.16, 0.49, 1.48, 4.44, 13.3 and 40. 10,000 cells suspended in 80 μl media were seeded into each well of a 96-well cell-culture plate and grown for 24 hours prior to treatment. 10 μl of 60 μg/mL heparin (Sigma Catalog # H3149) was added to each well, and then 10 μl of 50 μg/mL FGF2 (R&D Catalog #233-FB/CF) or media was added to each well of the plates. 10 μl of each of the compound dilutions described above and 10 μl of media were added to wells to a final volume 120 μl such that all pair-wise combinations as well as the single agents were represented. Cells were incubated for 72 hrs at 37° C. in a 5% CO2 incubator following compound addition. Cell proliferation was measured using the CellTiter-Glo luminescent cell viability assay (Promega catalog #G755B) and Victor4 plate reader (Perkin Elmer). Synergy scores and CI₇₀ calculations were determined as described elsewhere (Lehar J, Krueger A S, et al. Nat Biotechnol 2009; 27: 659-666).

### Western Blotting

Protein lysates were prepared from cell monalayers using RIPA buffer (Cell Signaling Technology Catalog #9806) according to the procedure described by the manufacturer. Antibodies to detect phospho-KIT (Catalog #3073S), total KIT (Catalog #3308), phospho-AKT S473 (Catalog #4058), total AKT (Catalog #9272), phospho-ERK (Catalog #9101), total ERK (Catalog #9107) and phospho-FRS2 (Catalog #3864) were purchased from Cell Signaling Technology. Antibody to GAPDH (Catalog #MAB374) was purchased from Millipore and anti-FRS2(H-91) (Catalog #sc-8318) from Santa Cruz. Bound antibody was detected using the LI-COR Odyssey Infrared Imaging System.

### RESULTS

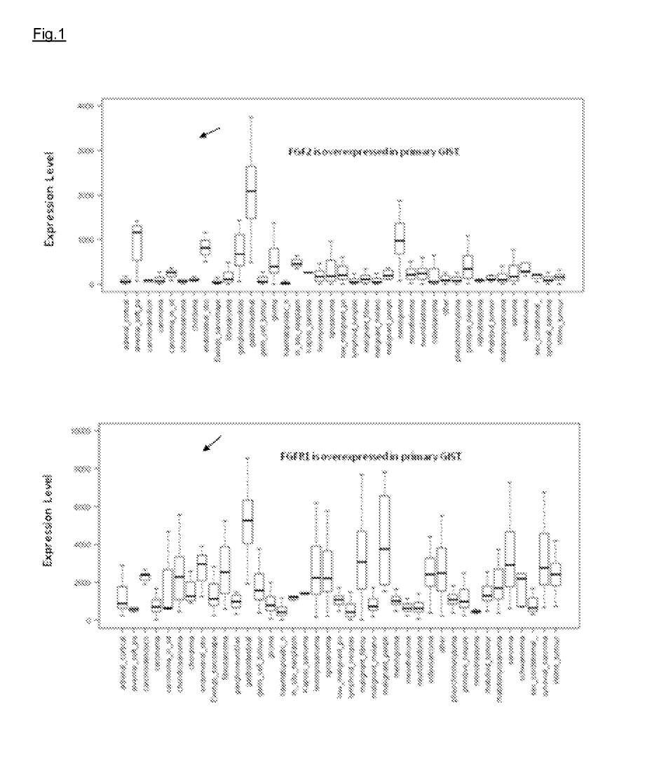

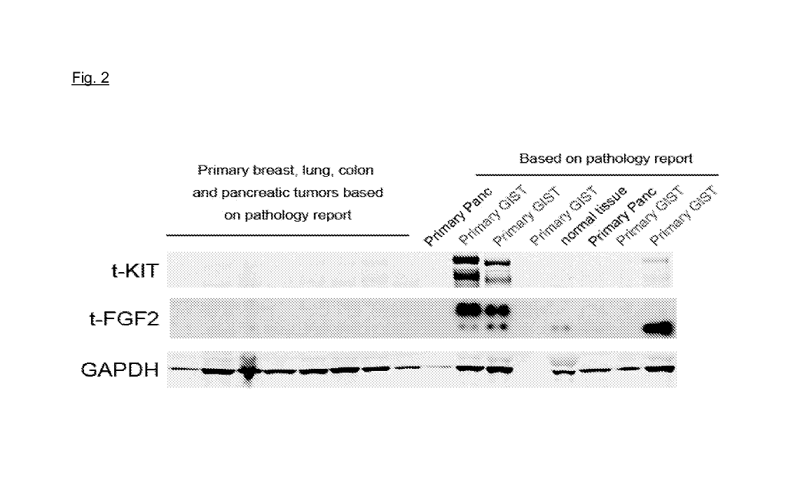

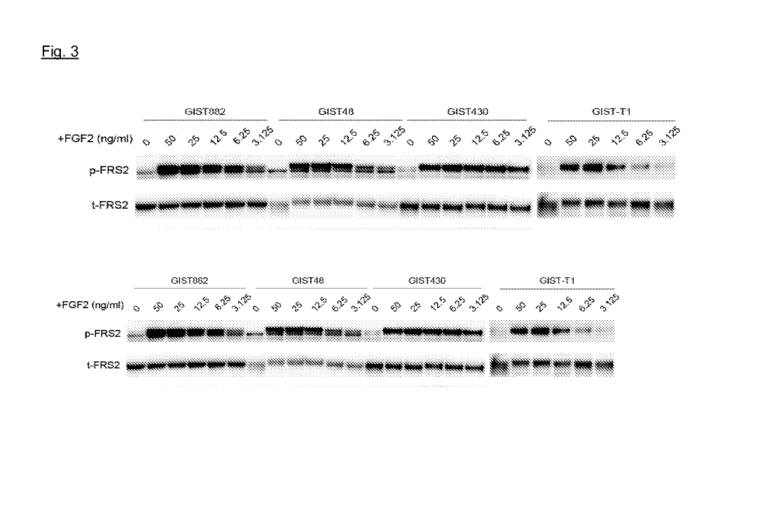

Novartis OncExpress database contains both internally and publically deposited expression data for 30,094 primary tumors, including 110 GIST samples, profiled by Affymetrix Human Genome U133A or U133 Plus 2.0 arrays. In addition to the known GIST-specific genes, such as KIT, ETV1 and PRKCQ, FGF2 and its receptor FGFR1 showed the highest average expression levels in GIST among 41 tumor types included in this dataset (FIG. 1), suggesting that FGFR pathway could be a survival pathway in GIST. FGF2 was also found to be over-expressed at the protein level in primary GISTs (FIG. 2). FGFR1, but not FGF2, is over-expressed in GIST cell lines. However, the FGFR signaling pathway was activated when various concentrations of exogenous FGF2 was added (FIG. 3).

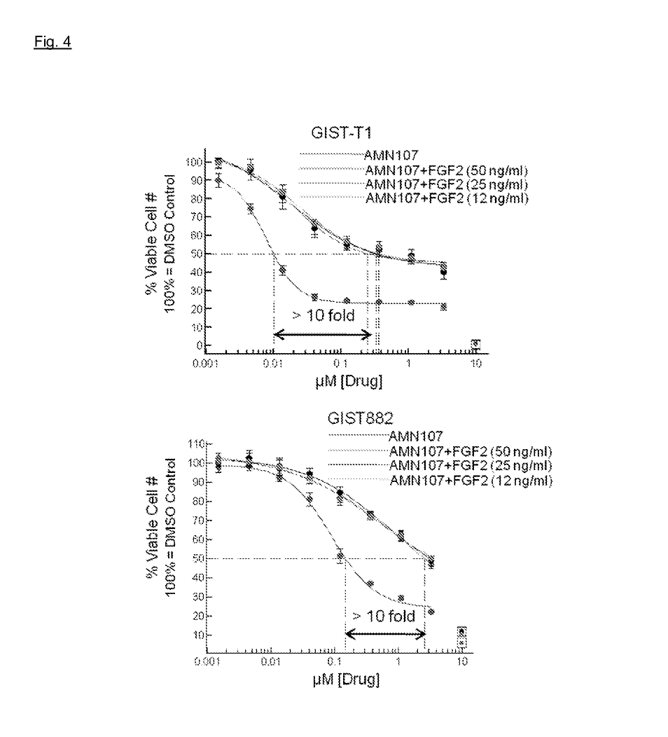

GIST-T1 and GIST882 are sensitive to KIT inhibition achieved by nilotinib (AMN107) treatment (FIG. 4). However, these two cell lines were shown to be less sensitive to KIT inhibition in the presence of added FGF2 with the GI₅₀ values shifted greater than 10 fold (FIG. 4), suggesting that FGFR signaling can function as a survival pathway once activated. Therefore, combining a KIT inhibitor and a potent FGFR inhibitor should enhance the growth inhibition in the GIST cell lines.

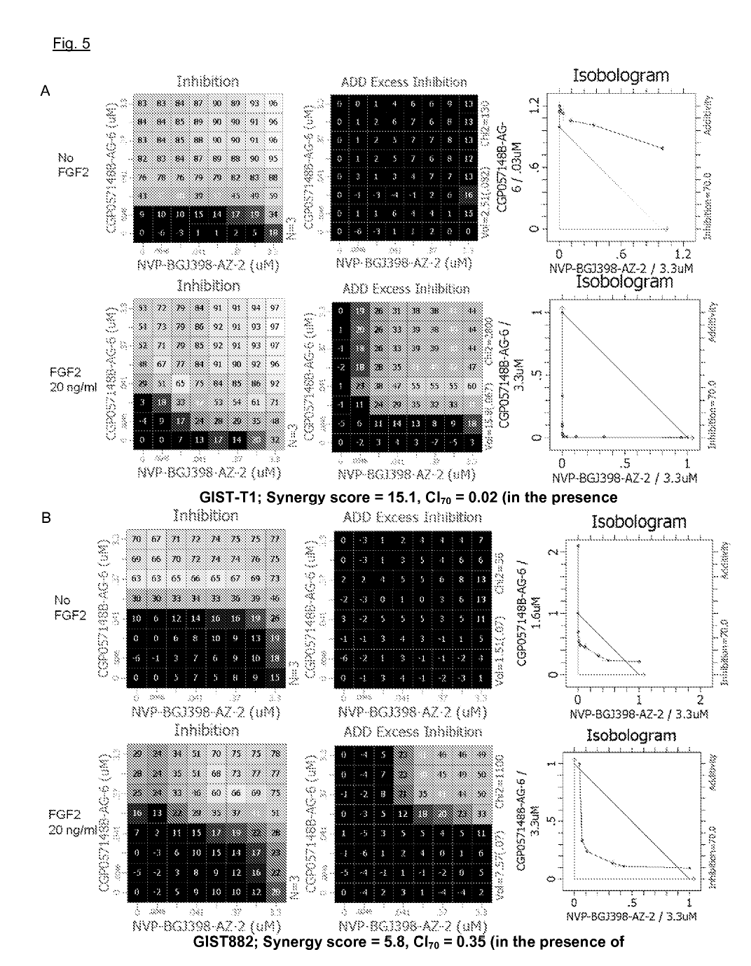

BGJ398 is an orally active, potent and selective inhibitor of FGFRs. To determine the single agent and combination effects of combining the FGFR inhibitor BGJ398 and the KIT inhibitor imatinib (CGP057148B) on the growth inhibition of GIST cells, we compared the proliferation responses for cells treated with dose ranges of each compound alone and pair-wise combinations for 3 days. As a single agent, imatinib was efficacious in inhibiting GIST-T1 and GIST882 growth in the absence of FGF2 (FIG. 5). In the presence of added FGF2, these two cell lines were less sensitive to imatinib treatment (FIG. 5), similar to the results shown in FIG. 4. BGJ398 did not significantly affect the viability of GIST cell lines, either in the presence or the absence of FGF2 (FIG. 5). However, BGJ398 combination with a KIT inhibitor (imatinib or nilotinib) resulted in strong combination effects in the presence of FGF2 in GIST cells. Combination effects were shown in FIG. 5 and determined by combination indices at a 70% inhibitory effect (CI₇₀) that measure dose shifting yielding 70% growth inhibition and by synergy scores that measure overall synergy observed across the entire dose matrixes (Lehar J, Krueger A S, al. Nat Biotechnol 2009; 27: 659-666).

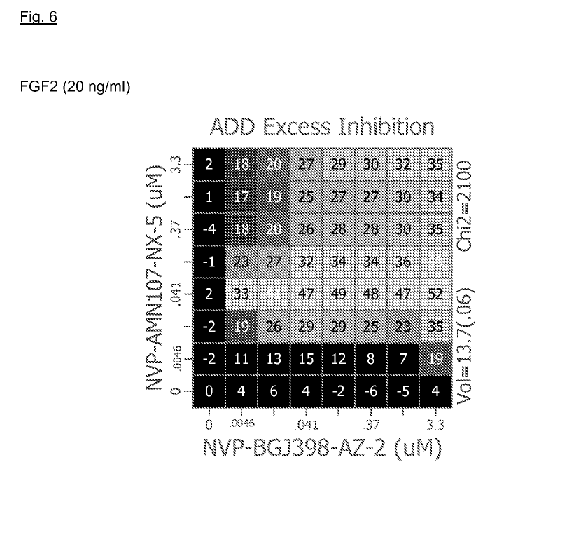

Also the combination of nilotinib and BGJ398 shows synergy in GIST cell lines even in the presence of FGF2 (FIG. 6).

### CONCLUSION

The expression profiles of more than 30,000 primary tumors show that FGF receptor 1 (FGFR1) and its ligand, FGF2, are highly expressed in primary GISTs, suggesting that the FGFR pathway is activated in GISTs. In addition, the FGFR pathway, when activated, can function as a survival pathway in GIST cell lines, making them less sensitive to KIT inhibition. However, combining FGFR inhibitors with KIT inhibitors resulted in strong synergistic and robust inhibition of the growth of GIST cell lines and restored complete growth inhibition by imatinib inhibition. These results suggest that a combination comprising an FGFR inhibitor and a KIT inhibitor can improve the current therapeutic strategy in GIST.

### Example 2

### Effects of Imatinib in Combination with PI3K Inhibitors on the Growth of GIST Cell Lines

The effects of COMPOUND A, COMPOUND C, COMPOUND D and of imatinib have been evaluated both as single agents and in combination in patient-derived GIST882 (expressing K642E mutant KIT), GIST48 (expressing V560D/D830A KIT), GIST430 (expressing ex11del/V654A KIT) and GIST-T1 (expressing ex11del KIT) cell lines. As single agents imatinib potently inhibited the proliferation of the GIST882, GIST430 and GIST-T1 cell lines (GIST48 being imatinib resistant) and COMPOUND A and COMPOUND C inhibited the proliferation of all four cell-lines at low micromolar concentrations, whereas COMPOUND D showed little or no effect on the proliferation of any of the cell-lines. When the antiproliferative effects of imatinib and COMPOUND A were evaluated in combination, growth suppression was observed in excess of the percent inhibition achieved by imatinib or COMPOUND A single agent treatment in GIST882 and GIST430 cell-lines. When the antiproliferative effects of imatinib and COMPOUND C were evaluated in combination, growth suppression was observed in excess of the percent inhibition achieved by imatinib or COMPOUND C single agent treatment in both the GIST882 and GIST430 cell-lines. When the antiproliferative effects of imatinib and COMPOUND D were evaluated in combination, growth suppression was observed in excess of the percent inhibition achieved by imatinib or COMPOUND D single agent treatment in the imatinib insensitive GIST48 and GIST430 cell-lines.

TABLE 1

| Imatinib com- | | | | |
|---|---|---|---|---|
| bined with | GIST882 | GIST-T1 | GIST48 | GIST430 |
| | Synergy Score for inhibition of GIST cell proliferation | | | |
| COMPOUND A | 5.07 | 1.10 | 0.38 | 2.06 |
| COMPOUND C | 1.90 | 1.29 | 1.20 | 1.96 |
| COMPOUND D | 0.46 | 1.06 | 1.98 | 2.04 |
| | Combination Index for 70% inhibition | | | |
| | of GIST cell proliferation | | | |
| COMPOUND A | 0.194 | 0.493 | 1.25 | 0.260 |
| COMPOUND C | 0.597 | 0.782 | 0.716 | 0.252 |
| COMPOUND D | — | 0.988 | 1.18 | 0.791 |

Synergy is quantified either as the ‘weighted’ Synergy Score, S (where S≦1 indicates either some additivity or no cooperativity or, S>1 suggests of some synergy and S>2 indicates significant synergy) or as Combination Indices, CI (where CI=1 indicates dose additivity, CI<0.5 indicates “real” synergy (2× dose shift), CI<0.3 indicates “useful” synergy (3× shift) and CI<0.1 indicates “strong” synergy (10× shift). Significant assessments of synergy are indicated in bold-type.

### Example 3

### Single-Arm Dose-Finding Phase Ib Study of Imatinib in Combination with the Oral Phosphatidyl-Inositol 3-Kinase (PI3-K) Inhibitor COMPOUND C in Patients with Gastrointestinal Stromal Tumor (GIST) Who Failed Prior Therapy with Imatinib and Sunitinib

### Inclusion Criteria:

1. Male or female patients ≧18 years of age
2. WHO performance status (PS) of 0-2
3. Histologically confirmed diagnosis of GIST that is unresectable or metastatic
4. Available tissue specimen:
   Dose-escalation cohorts: patients must have available archival tumor tissue which can be shipped during the course of the study
   Dose-expansion cohort: patients must have available archival tumor tissue which can be shipped during the course of the study and must agree to a fresh pretreatment biopsy.
5. Failed prior therapy with imatinib followed by sunitinib for the treatment of unresectable or metastatic GIST. Note the following specific criteria for the two phases of the trial:
   Dose-escalation cohorts: patients who failed prior therapy with imatinib and then have failed therapy with sunitinib. Treatment failure may be due to either disease progression on therapy (both imatinib and sunitinib) or intolerance to therapy (sunitinib).
   Dose-expansion cohort: patients must have documented disease progression on both imatinib and sunitinib. In addition, patients may have had no more than two lines of prior therapy (i.e. treatment with imatinib followed by treatment with sunitinib).

1. Method of treating GIST in a human patient comprising administering to the human patient in need thereof a dose effective against GIST of a combination (a) a c-kit inhibitor and (b) a PI3K inhibitor or FGFR inhibitor, or a pharmaceutically acceptable salt thereof, respectively.

2. Method according to claim 1, wherein the c-kit inhibitor is selected from imatinib, nilotinib and masitinib, or, respectively, a pharmaceutically acceptable salt thereof.

3. Combination comprising (a) a c-kit inhibitor and (b) a PI3K inhibitor or FGFR inhibitor, or a pharmaceutically acceptable salt thereof, respectively, for the treatment of GIST.

4. Method according to claim 1 or combination according to claim 3, wherein the GIST is progressing after imatinib therapy.

5. Method according to claim 1 or combination according to claim 3, wherein the GIST is progressing after imatinib and sunitinib therapy.

6. Method according to claim 2, wherein imatinib is applied in a daily dose between 300 and 600 mg.

7. Method according to claim 1 or combination according to claim 3, wherein the PI3K inhibitor is selected from 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, and (S)-pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), or, respectively, a pharmaceutically acceptable salt thereof.
